(19) United States
(12) Patent Application Publication
(10) Pub. No.: US 2026/0243737 A1
(43) Pub. Date: Aug. 20, 2026

(54) APPARATUS AND METHOD FOR ANALYZING AND MANAGING HYDROGEN QUALITY

(71) Applicants: Hyundai Motor Company, Seoul (KR); Kia Corporation, Seoul (KR)

(72) Inventors: Seong Chul AHN, Hwaseong-Si (KR); Yeon Jong RYU, Hwaseong-Si (KR); Hyun Seung KIM, Hwaseong-Si (KR); Pil Seon HEO, Hwaseong-Si (KR); Choong Hoon JEONG, Hwaseong-Si (KR)

(21) Appl. No.: 19/296,356

(22) Filed: Aug. 11, 2025

(30) Foreign Application Priority Data
Feb. 14, 2025 (KR) 10-2025-0019569

(51) Int. Cl. G01N 30/20; G01N 30/00; G01N 30/02; G01N 33/00

(52) U.S. Cl. CPC G01N 30/20; G01N 33/0006; G01N 33/0016; G01N 2030/0095; G01N 2030/025

(57) ABSTRACT

An apparatus and a method may be used for analyzing and managing hydrogen quality. The apparatus includes a first gas analyzer configured to analyze the concentration of a plurality of impurity components contained in a target gas including hydrogen to be analyzed using first and second laser analysis methods, a second gas analyzer configured to analyze the concentration of the plurality of impurity components using a non-laser analysis method, and a quality determination unit configured to determine the suitability of quality of the target gas based on the results of the analysis by the first and second gas analyzers.

### CROSS-REFERENCE TO RELATED APPLICATIONS

This application claims the benefit of Korean Patent Application No. 10-2025-0019569, filed on Feb. 14, 2025, which is hereby incorporated by reference as if fully set forth herein.

### TECHNICAL FIELD

This disclosure relate to an apparatus and a method for analyzing and managing hydrogen quality.

### BACKGROUND

In response to carbon neutrality and climate change, hydrogen vehicles are expected to play a key role in mobility applications (e.g., vehicles), and hydrogen consumption is expected to increase. However, the establishment of the hydrogen ecosystem and infrastructure is still at an early stage. Efforts at the national level are required to promote widespread adoption of hydrogen vehicles. To this end, it is important to secure a stable supply of hydrogen and to ensure reliable hydrogen quality.

Charging a hydrogen vehicle using low-quality hydrogen (e.g., that does not satisfy the required quality specifications) may cause failure of the fuel cell of the hydrogen vehicle. Failure of the fuel cell may result in the hydrogen vehicle breaking down and/or malfunctioning during operation. While quality control of hydrogen is critically important, there is no method for real-time hydrogen quality analysis and management. Accordingly, when a hydrogen quality issue occurs, immediate/timely countermeasures may not be taken, and only post-incident responses may be available, potentially resulting in greater damage.

In the case of on-site hydrogen refueling stations producing hydrogen in real time and refueling vehicles with hydrogen, such damage may be particularly serious. As a result, such issues may cause substantial harm not only to businesses involved in hydrogen production and infrastructure, but also to hydrogen vehicle manufacturers and consumers. These harms may hinder widespread adoption of hydrogen vehicles and establishment of the hydrogen ecosystem.

The matters described in this Background section are only for enhancement of understanding of the background of the disclosure, and should not be taken as acknowledgement that they correspond to prior art already known to those skilled in the art.

### SUMMARY OF THE DISCLOSURE

The following summary presents a simplified summary of certain features. The summary is not an extensive overview and is not intended to identify key or critical elements.

Systems, apparatuses, and methods are described for analyzing and managing hydrogen quality. An apparatus may comprise: a first gas analyzer comprising at least one laser absorption spectrometer configured to: measure one or more first concentrations of one or more first impurity components in a target gas comprising hydrogen; and measure one or more second concentrations of one or more second impurity components in the target gas; a second gas analyzer configured to measure, via a non-laser-based sensing method, one or more third concentrations of one or more third impurity components in the target gas; and a controller comprising: a processor; and a memory storing instructions that, when executed by the processor, are configured to cause the apparatus to: receive, from the first gas analyzer, first information associated with the one or more first concentrations and second information associated with the one or more second concentrations; and receive, from the second gas analyzer, third information associated with the one or more third concentrations; determine, based on the first information, the second information and the third information, an indicator of a quality of the target gas; and control, based on the determined indicator of the quality, a process associated with supply of hydrogen.

Also, or alternatively, an apparatus may comprise: a first gas selection valve system configured to select a target gas comprising hydrogen to be analyzed, wherein the target gas is collected at one or more stages of a process associated with supply of hydrogen, wherein the process comprises stages of hydrogen production, compressed hydrogen storage, hydrogen backup supply, and hydrogen charging; a plurality of gas analyzers configured to measure concentrations of a plurality of impurity components in the target gas selected by the first gas selection valve system; and a control circuit configured to cause the apparatus to: receive, from the plurality of gas analyzers, information indicating one or more concentrations of one or more impurity components of the plurality of impurity components; determine, based on the information, an indicator of a quality of the target gas; and control, based on the determined indicator of the quality, the process associated with the supply of hydrogen.

A method performed by an apparatus (e.g., disclosed herein) for analyzing and managing hydrogen quality may comprise: controlling flow of a target gas, of a plurality of target gases, to a plurality of gas analyzers configured to measure concentrations of a plurality of impurity components, wherein the plurality of target gases comprises hydrogen to be analyzed and each target gas of the plurality of target gases is collected at a different stage of a process associated with hydrogen supply, wherein the process comprises stages of hydrogen production, compressed hydrogen storage, hydrogen backup supply, and hydrogen charging; receiving, from the plurality of gas analyzers, information indicating one or more concentrations of one or more impurity components of the plurality of impurity components; determining, based on the information, an indicator of a quality of the target gas; and controlling, based on the determined indicator of the quality, the process associated with the supply of hydrogen.

The hydrogen quality analysis and management apparatus and method disclosed herein may efficiently and reliably analyze the quality of hydrogen (e.g., in real time) and manage the quality of hydrogen using the results of the analysis, thereby enabling stable supply of hydrogen.

These and other features and advantages are described in greater detail below.

### BRIEF DESCRIPTION OF THE DRAWINGS

The accompanying drawings, which are included to provide a further understanding of the disclosure and are incorporated in and constitute a part of this application, illustrate example(s) of the disclosure and together with the description serve to explain the principle of the disclosure. In the drawings:

FIG. 1 is a block diagram of a hydrogen quality analysis and management apparatus according to an example;

FIG. 2 is a flowchart for explaining a hydrogen quality analysis and management method according to an example;

FIG. 3 is a block diagram of an example of the target selection unit shown in FIG. 1;

FIG. 4 is a block diagram showing a hydrogen processing apparatus;

FIG. 5 is a block diagram of an example of the standard gas supply unit shown in FIG. 1;

FIG. 6 is a block diagram of an example of the explosion-prevention unit shown in FIG. 1; and

FIG. 7 is a front view showing the external appearance of the hydrogen quality analysis and management apparatus according to the example.

### DETAILED DESCRIPTION OF THE DISCLOSURE

The present disclosure will now be described more fully hereinafter with reference to the accompanying drawings, in which various examples are shown. The examples, however, may be embodied in many different forms, and should not be construed as being limited to the examples set forth herein. Rather, these examples are provided so that this disclosure will be more thorough and complete, and will more fully convey the scope of the disclosure to those skilled in the art.

It will be understood that an element referred to as being “on” or “under” another element may be directly on or under the other element, or one or more intervening elements may also be present. Disclosure of an element “on” or “under” another element includes disclosure of the other element “under the element” as well as “on the element.”.

Relational terms, such as “first”, “second”, “on/upper part/above”, and “under/lower part/below”, are used only to distinguish between one subject or element and another subject or element, without necessarily requiring or involving any physical or logical relationship or sequence between the subjects or elements. “On/upper part/above”, and “under/lower part/below” may refer to relative positions as depicted in the figures, for example, but may not require an absolute orientation (e.g., relative to the ground).

For purposes of this application and the claims, using the exemplary phrase “at least one of: A; B; or C” or “at least one of A, B, or C,” the phrase means “at least one A, or at least one B, or at least one C, or any combination of at least one A, at least one B, and at least one C. Further, exemplary phrases, such as “A, B, or C”, “at least one of A, B, and C”, “at least one of A, B, or C”, etc. as used herein may mean each listed item or all possible combinations of the listed items. For example, “at least one of A or B” may refer to (1) at least one A; (2) at least one B; or (3) at least one A and at least one B. “One or more of” is synonymous with “at least one of” herein.

The term “about” in relation to a reference numerical value, and its grammatical equivalents as used herein, can include the reference numerical value itself and a range of values plus or minus 10% from that reference numerical value. For example, the term “about 10” includes 10 and any amount from and including 9 to 11. In some cases, the term “about” in relation to a reference numerical value can also include a range of values plus or minus 10%, 9%, 8%, 7%, 6%, 5%, 4%, 3%, 2%, or 1% from that reference numerical value. In some embodiments, “about” in connection with a number or range measured by a particular method indicates that the given numerical value includes values determined by the variability of that method.

Unless otherwise defined, the terms used herein, including technical or scientific terms, may have meanings generally understood by those skilled in the art to which the present disclosure belongs.

The expressions such as “comprise”, “may comprise”, “include”, “may include”, “have”, “may have”, etc. as used herein are intended to mean the presence of a characteristic (e.g., function, operation, component, etc.) and do not exclude the presence of other additional characteristics. That is, these expressions should be understood as open-ended terms that encompass the possibility that other examples are included.

A singular expression used herein may include the meaning of the plural unless otherwise stated in the context, which also applies to the singular expression described in the claims.

The expression “based on” as used herein is intended to describe one or more factors that influence an act or operation of determining or deciding described in a phrase or sentence including that expression, and this expression does not exclude any additional factors that influence the act or operation of determining or deciding.

Depending on the context, the expression “configured to” as used herein may have meanings such as “set to”, “with the ability to”, “modified to”, “made to”, “to be able to”, etc. This expression is not limited to the meaning of “specially designed in hardware to”. For example, a processor configured to perform a specific operation may refer to a generic purpose processor capable of performing the specific operation by executing software, or to a special purpose computer structured through programming to perform the specific operation.

Hereinafter, an apparatus and a method for analyzing and managing hydrogen quality according to examples will be described.

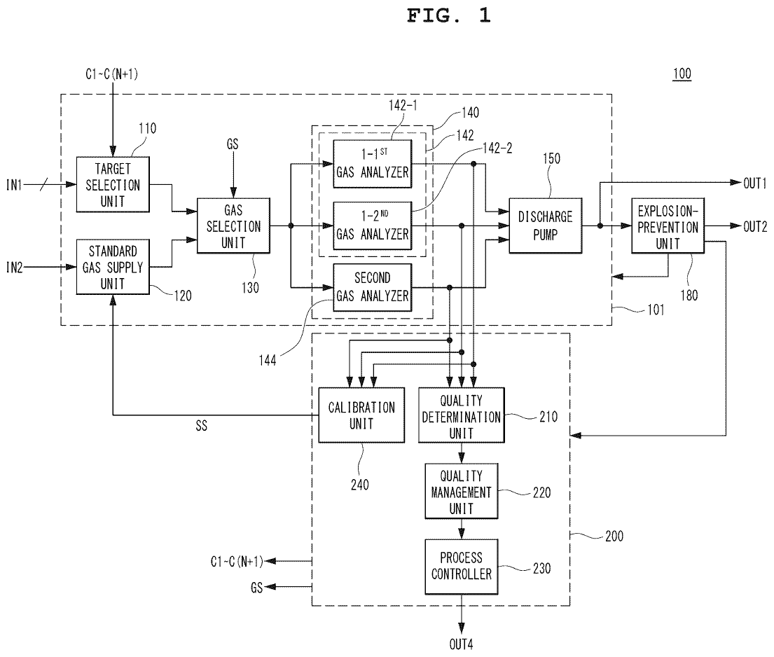

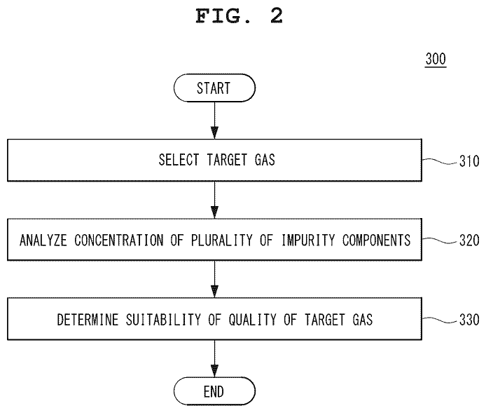

FIG. 1 is a block diagram of a hydrogen quality analysis and management apparatus 100 according to an example, and FIG. 2 is a flowchart for explaining a hydrogen quality analysis and management method 300 according to an example.

Hereinafter, the hydrogen quality analysis and management apparatus 100 shown in FIG. 1 will be described as performing the hydrogen quality analysis and management method 300 shown in FIG. 2. However, the examples are not limited thereto. That is, the hydrogen quality analysis and management apparatus 100 shown in FIG. 1 may perform a method different from the hydrogen quality analysis and management method 300 shown in FIG. 2, and the hydrogen quality analysis and management method 300 shown in FIG. 2 may be performed by a hydrogen quality analysis and management apparatus configured differently from the hydrogen quality analysis and management apparatus 100 shown in FIG. 1.

The hydrogen quality analysis and management apparatus 100 shown in FIG. 1 may include a gas analysis unit 140 (e.g., gas analyzer/gas concentration sensor) and a controller 200. The hydrogen quality analysis and management apparatus 100 may include a target selection unit (e.g., a pipe branching unit, first gas selection valve system) 110. The hydrogen quality analysis and management apparatus 100 may include a standard gas supply unit 120 (e.g., a standard gas supply) and a gas selection unit 130 (e.g., second gas selection valve system). The hydrogen quality analysis and management apparatus 100 may include a discharge pump 150. The hydrogen quality analysis and management apparatus 100 may include an explosion-prevention unit 180.

The gas analysis unit 140 may analyze the concentration of one or more (e.g., a plurality of) impurity components (hereinafter referred to as “components”) potentially contained in a gas including hydrogen to be analyzed (hereinafter referred to as “target gas”) (step 320). For example, the gas containing the hydrogen to be analyzed may contain one or more of a plurality of impurity components, and the gas analysis unit 140 may be configured to detect any of the plurality of impurity components that may be in the hydrogen to be analyzed (e.g., target gas). The gas analysis unit 140 may analyze/measure/determine the concentration of the detected impurity component(s)

According to the example, the gas analysis unit 140 may analyze the concentration of the one or more components using various methods. For example, the gas analysis unit 140 may analyze the concentration of the plurality of components by combining a first laser analysis, a second laser analysis method and/or a non-laser analysis method.

To this end, the gas analysis unit 140 may include a first gas analyzer 142 and a second gas analyzer 144.

The first gas analyzer 142 (e.g., a laser-based gas analyzer, a laser absorption spectroscopy device/spectrometer) may analyze the target gas using the first and second laser analysis methods (e.g., laser absorption methods). The first gas analyzer 142 may include a 1-1^{st} gas analyzer 142-1 and a 1-2^{nd} gas analyzer 142-2. The first and second laser analysis methods may be the same or different laser absorption techniques, but may be optimized/calibrated to detect/analyze concentrations of different subsets of the plurality of impurity components.

The 1-1st gas analyzer 142-1 may analyze the concentration of a first set of components (e.g., CO₂, NH₃, HCOOH, HCHO, CO, and CH₄), among the plurality of components, using optical feedback cavity enhanced absorption spectroscopy (OFCEAS) as the first laser analysis method. For example, the 1-1st gas analyzer 142-1 may include two cells, one configured to measure the concentration of CO₂, NH₃, and HCOOH and the other configured to measure the concentration of HCHO, CO, and CH₄.

The 1-2^{nd} gas analyzer 142-2 may analyze the concentration of a second set of the components (e.g., H₂S and H₂O), among the plurality of components, using OFCEAS as the second laser analysis method. For example, the 1-2^{nd} gas analyzer 142-2 may include one cell configured to measure the concentration of H₂S and H₂O.

According to another example, the laser analysis method is not limited to the above-described first and second laser analysis methods and may be modified in various forms through combinations of analysis cells of the laser analyzer.

For example, the components to be analyzed may be classified according to three or more laser analysis methods, and the combinations of the components analyzed by the 1-1st gas analyzer 142-1 and the 1-2^{nd} gas analyzer 142-2 may differ from those described above.

The second gas analyzer 144 may analyze the target gas using a non-laser analysis method. For example, using gas chromatography (GC) as the non-laser analysis method, the second gas analyzer 144 (e.g., gas chromatograph) may analyze the concentration of a third set of components (e.g., N₂ and O₂) among the plurality of components.

According to the example described above, the gas analysis unit 140 may analyze the concentration of components in the first, second and third sets of components, for example, ten impurity components potentially contained in the target gas, such as, CO₂, NH₃, HCOOH, HCHO, CO, CH₄, H₂S, H₂O, N₂, and O₂.

The aforementioned ten impurity components are items that are highly likely to be detected in the target gas according to ISO 14687 and that significantly affect fuel cell apparatuses using hydrogen, such as hydrogen fuel cell vehicles.

For example, the target gas analyzed by the gas analysis unit 140 may be selected at any time point during a process of preparing and/or supplying the hydrogen (e.g., for use). For example, the process herein may comprise one or more of hydrogen production, hydrogen storage (e.g., after compression), hydrogen backup supply, and/or charging. That is, the target selection unit 110 may receive the target gas, discharged at certain time points during the process mentioned above, via an input terminal IN1. The target selection unit 110 may select at least one of the received target gases, and may discharge the selected target gas (step 310).

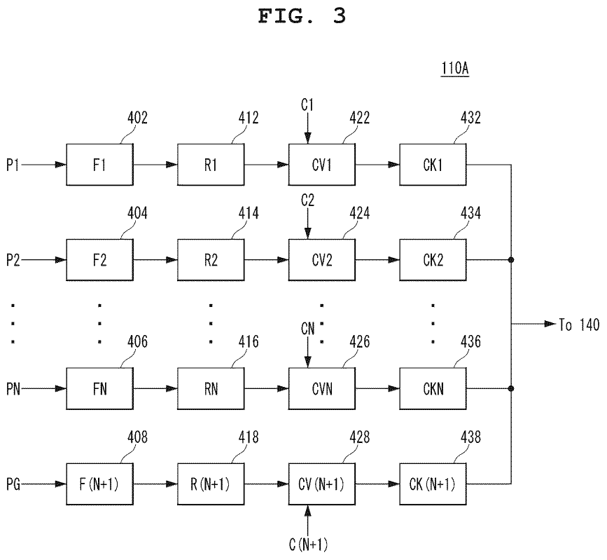

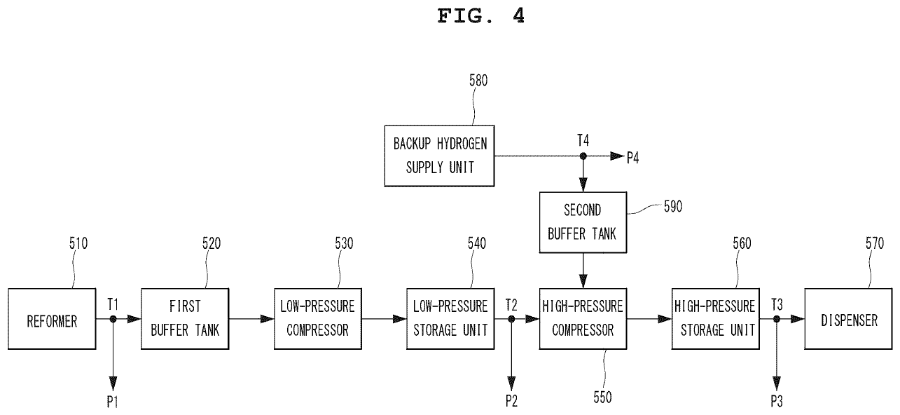

FIG. 3 is a block diagram of an example 110A of the target selection unit 110 shown in FIG. 1, and FIG. 4 is a block diagram showing a hydrogen processing apparatus.

Referring to FIG. 4, a hydrogen processing apparatus may include a reformer 510, a first buffer tank 520, a low-pressure compressor 530, a low-pressure storage unit 540, a high-pressure compressor 550, a high-pressure storage unit 560, a dispenser 570, and/or a backup hydrogen supply unit 580. The hydrogen processing apparatus may or may not include a second buffer tank 590.

The reformer 510 may produce hydrogen and supply the produced hydrogen to the first buffer tank 520. The first buffer tank 520 may store the produced hydrogen.

The low-pressure compressor 530 may compress the hydrogen stored in the first buffer tank 520 to a first pressure (e.g., low relative to a second pressure of hydrogen stored in the high-pressure storage unit 560). In order to store the hydrogen in a hydrogen transport tank configured to transport hydrogen, the hydrogen may be compressed to the first pressure (e.g., a low/safe pressure for transport). The low-pressure storage unit 540 may store the hydrogen compressed to the first pressure. The hydrogen stored in the low-pressure storage unit 540 may be transferred and stored in a hydrogen storage tank of a tanker truck, for example. The high-pressure compressor 550 may compress the hydrogen stored in the low-pressure storage unit 540 to a second pressure (e.g., high pressure, higher pressure than the first pressure). Since hydrogen vehicles require hydrogen compressed to a high pressure rather than a low pressure, the hydrogen may be compressed to the second pressure (e.g., a high pressure) by the high-pressure compressor 550. The high-pressure storage unit 560 may store the hydrogen compressed to a high pressure. The dispenser 570 serves to charge the hydrogen, (e.g., compressed to the second pressure and stored in the high-pressure storage unit 560) into a hydrogen vehicle.

The backup hydrogen supply unit 580 may comprise a tanker truck that carries hydrogen produced elsewhere and/or equipment to which hydrogen produced elsewhere is supplied via pipelines. The second buffer tank 590 may store the hydrogen supplied from the backup hydrogen supply unit 580 and/or may supply the stored hydrogen to the high-pressure compressor 550.

Hydrogen discharged at points T1, T2, T3, and/or T4 during the process (e.g., shown in FIG. 4) may be supplied, as target hydrogen, to the target selection unit 110 (e.g., shown in FIG. 1). The points may be collected from different locations corresponding to the different process steps/stages as discussed herein. The target hydrogen from the different points may be collected at the same or different times. The target hydrogen from the different points may each be supplied to the target selection unit 110 via individual pipes.

The target gas introduced into the target selection unit 110 may include N target gases. For example, N may be a positive integer of 2 or greater. For example, N may be 4, and the plurality of target gases may include at least two of first to fourth target gases.

The first target gas may include hydrogen produced by the reformer 510. The second target gas may include hydrogen compressed to the first pressure (e.g., after being produced) and may be discharged from the low-pressure storage unit 540. The third target gas may include hydrogen compressed to the second pressure (e.g., after being compressed from the pressure) and may be discharged from the high-pressure storage unit 560. The fourth target gas may include backup hydrogen and may be discharged from the backup hydrogen supply unit 580.

The hydrogen quality analysis and management apparatus 100, according to the example, may include 1st to N^{th} pipes P1 to PN or may be connected to the first to N^{th} pipes P1 to Pn. Herein, “i” refers to an integer from 1 to N. An i^{th} pipe Pi may be a pipe through which an i^{th} target gas flows. For example, when N is 4, the hydrogen quality analysis and management apparatus 100 according to the example may include first to fourth pipes P1 to P4 or may be connected to the first to fourth pipes P1 to P4.

The first pipe P1 may be connected between the reformer 510, which produces hydrogen, and the target selection unit 110 to define a flow path through which the first target gas flows.

The second pipe P2 may be connected between the low-pressure storage unit 540, which stores the hydrogen produced by the reformer 510 and compressed to the first pressure, and the target selection unit 110 to define a flow path through which the second target gas flows.

The third pipe P3 may be connected between the high-pressure storage unit 560, which stores the hydrogen compressed to a high pressure, and the target selection unit 110 to define a flow path through which the third target gas flows.

The fourth pipe P4 may be connected between the backup hydrogen supply unit 580 and the target selection unit 110 to define a flow path through which the fourth target gas flows.

The target selection unit 110 may select at least one of the first to N^{th} target gases and/or may sequentially and/or alternately discharge the selected target gases to the gas analysis unit 140.

For example, as shown in FIG. 3, the target selection unit 110A may include first to N^{th} filters (F1 to FN) 402, 404, . . . , and 406, first to N^{th} regulators (R1 to RN) 412, 414, . . . , and 416, first to N^{th} control valves (CV1 to CVN) 422, 424, . . . , and 426, and first to N^{th} check valves (CK1 to CKN) 432, 434, . . . , and 436.

The i^{th} filter Fi may filter contaminants contained in the i^{th} target gas and may discharge the filtered i^{th} target gas to the i^{th} regulator Ri.

The i^{th} regulator Ri may regulate the pressure of the i^{th} target gas filtered by the i^{th} filter Fi and/or may discharge the i^{th} target gas having the regulated pressure to the i^{th} control valve CVi.

The i^{th} control valve CVi may allow and/or restrict the flow of the n^{th} target gas output from the i^{th} regulator Ri in response to an i^{th} opening/closing control signal Ci.

The i^{th} check valve CKi may prevent backflow of the i^{th} target gas toward the i^{th} control valve CVi.

The first to N^{th} target gases respectively discharged from the first to N^{th} check valves (CK1 to CKN) 432, 434, . . . , and 436 may be supplied to the gas analysis unit 140 via a single pipe (e.g., one or more shared pipes). If one of the first to N^{th} target gases is supplied to the gas analysis unit 140 and then another of the first to N^{th} target gases is immediately supplied to the gas analysis unit 140, the pipe through which the other target gas is supplied may be contaminated by the target gas previously supplied to the gas analysis unit 140.

In order to prevent this, according to an example, after one of the first to N^{th} target gases is discharged to the gas analysis unit 140, a purge gas PG (e.g., nitrogen N₂) may be discharged to the gas analysis unit 140 before another of the first to N^{th} target gases is discharged to the gas analysis unit 140 for analysis.

The target selection unit 110A may further include an N+1^{th} filter (F(N+1)) 408, an N+1^{th} regulator (R(N+1)) 418, an N+1^{th} control valve (CV(N+1)) 428, and an N+1^{th} check valve (CK(N+1)) 438.

The N+1^{th} filter (F(N+1)) 408 may filter contaminants contained in the purge gas PG and may supply the filtered purge gas to the N+1^{th} regulator (R(N+1)) 418.

The N+1^{th} regulator (R(N+1)) 418 may regulate the pressure of the purge gas filtered by the N+1^{th} filter (F(N+1)) 408 and may supply the purge gas having the regulated pressure to the N+1^{th} control valve (CV(N+1)) 428.

The N+1^{th} control valve (CV(N+1)) 428 may allow and/or restrict the flow of the purge gas output from the N+1^{th} regulator (R(N+1)) 418 (e.g., in response to an N+1^{th} opening/closing control signal C(N+1)).

The N+1^{th} check valve (CK(N+1)) 438 may prevent backflow of the purge gas PG toward the N+1^{th} control valve (CV(N+1)) 428.

According to an example, after one of the first to N^{th} target gases is supplied to the gas analysis unit 140, the purge gas may be supplied to the gas analysis unit 140 (e.g., before another of the first to N^{th} target gases is supplied to the gas analysis unit 140 for analysis).

For example, the controller 200 may generate first to fifth opening/closing control signals C1 to C5. The first to fifth opening/closing control signals C1 to C5 may cause the first to fourth target gases to be sequentially discharged from the target selection unit 110.

The controller 200 may allow the flow of the first target gas using the first opening/closing control signal C1 and/or may restrict the flow of the second to fourth target gases and the purge gas using the second to fifth opening/closing control signals C2 to C5.

The controller 200 may (e.g., subsequently) allow the flow of the purge gas using the fifth opening/closing control signal C5 and/or may restrict the flow of the first to fourth target gases using the first to fourth opening/closing control signals C1 to C4.

The controller 200 may (e.g., subsequently) allow the flow of the second target gas using the second opening/closing control signal C2 and/or may restrict the flow of the first, third, and fourth target gases and the purge gas using the first and third to fifth opening/closing control signals C1 and C3 to C5.

The controller 200 may (e.g., subsequently) allow the flow of the purge gas using the fifth opening/closing control signal C5 and/or may restrict the flow of the first to fourth target gases using the first to fourth opening/closing control signals C1 to C4.

The controller 200 may (e.g., subsequently) allow the flow of the third target gas using the third opening/closing control signal C3 and/or may restrict the flow of the first, second, and fourth target gases and the purge gas using the first, second, fourth, and fifth opening/closing control signals C1, C2, C4, and C5.

The controller 200 may (e.g., subsequently) allow the flow of the purge gas using the fifth opening/closing control signal C5 and/or may restrict the flow of the first to fourth target gases using the first to fourth opening/closing control signals C1 to C4.

The controller 200 may (e.g., subsequently) allow the flow of the fourth target gas using the fourth opening/closing control signal C4 and/or may restrict the flow of the first to third target gases and the purge gas using the first to third and fifth opening/closing control signals C1 to C3 and C5.

The standard gas supply unit 120 may receive a plurality of standard gases, each having standard concentration of a plurality of components, through an input terminal IN2. The standard gas supply unit 120 may supply the standard gases to the gas analysis unit 140. If the target gas includes a plurality of components (e.g., ten components, such as discussed herein), the standard gas supply unit 120 may supply standard gases corresponding to the plurality of components (e.g., CO₂, NH₃, HCOOH, HCHO, CO, CH₄, H₂S, H₂O, N₂, and O₂) to the gas analysis unit 140.

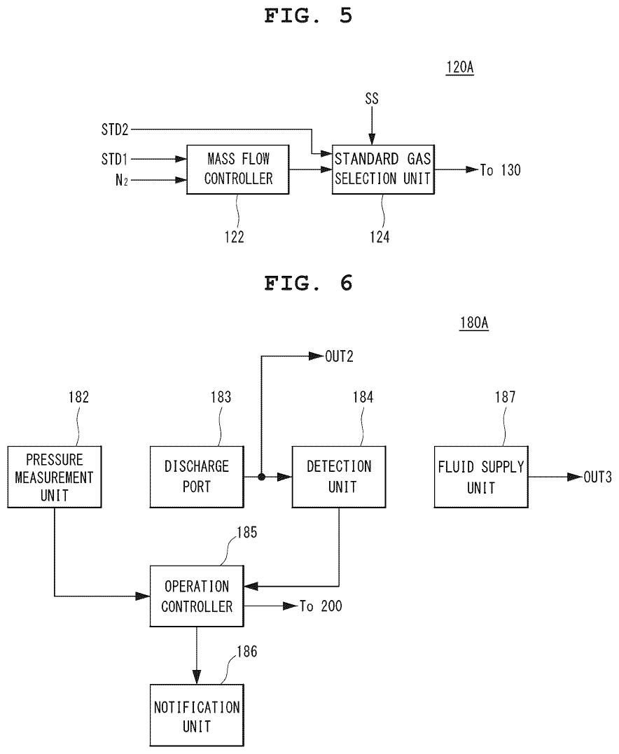

FIG. 5 is a block diagram of an example 120A of the standard gas supply unit 120 (e.g., as shown in FIG. 1).

The standard gas supply unit 120A may include a mass flow controller (MFC) 122 and a standard gas selection unit 124. The MFC 122 may adjust the flow rate and/or concentration of a first standard gas STD1. The MFC 122 may discharge the first standard gas having the adjusted flow rate and concentration. For example, the MFC 122 may adjust the concentration of the first standard gas STD1, such as oxygen (O₂), by diluting the same with nitrogen (N₂).

The standard gas selection unit 124 may select, in response to a selection signal SS (e.g., from the controller 200), a second standard gas STD2 (e.g., such as nitrogen (N₂) or oxygen (O₂)) whose flow rate and/or concentration have not been adjusted, or the first standard gas STD1 (e.g., having the flow rate and concentration adjusted by the MFC 122). The standard gas selection unit 124 may supply the selected gas to the gas selection unit 130 (e.g., gas selection valve). For example, the second standard gas STD2 selected by the standard gas selection unit 124 may be selected by the gas selection unit 130 and supplied to the second gas analyzer 144, and the first standard gas STD1, having the adjusted flow rate and concentration and selected by the standard gas selection unit 124, may be supplied to the first gas analyzer 142.

The gas selection unit 130 may supply an output from either the target selection unit 110 or the standard gas supply unit 120 to the gas analysis unit 140 (e.g., in response to a selection control signal GS, such as from the controller 200).

The discharge pump 150 may pump the target gas analyzed by the gas analysis unit 140 and/or may discharge the pumped target gas via an output terminal OUT1. The discharge pump 150 may pump and/or discharge the target gas (e.g., analyzed by the first and second gas analyzers 142 and 144), for example, so as to form a vacuum inside the first gas analyzer 142.

Also, or alternatively, the second gas analyzer 144 may discharge the target gas without passing through the discharge pump 150.

The hydrogen quality analysis and management apparatus 100 may further include a chamber (and/or an explosion-prevention enclosure (rack)) 101. For example, at least part of the apparatus 100 may be disposed in the chamber.

The chamber 101 may serve as a space in which at least one of the gas analysis unit 140, the target selection unit 110, the standard gas supply unit 120, the gas selection unit 130, and/or the discharge pump 150 is disposed. The chamber 101 may provide an environment that satisfies the explosion-prevention conditions of the apparatus 100 (e.g., shown in FIG. 1). For example, only the gas analysis unit 140 may be disposed in the chamber 101. The gas analysis unit 140, the target selection unit 110, the standard gas supply unit 120, the gas selection unit 130, and/or the discharge pump 150 may be disposed in the chamber 101.

The explosion-prevention unit 180 may inspect explosion factors in the chamber 101 and/or may perform operations associated with the explosion factors in response to the inspection results.

FIG. 6 is a block diagram of an example 180A of the explosion-prevention unit 180 shown in FIG. 1.

The explosion-prevention unit 180A may include at least one of a pressure measurement unit 182, a discharge port 183, a detection unit 184, an operation controller 185, a notification unit 186, and/or a fluid supply unit 187.

The pressure measurement unit 182 (e.g., pressure sensor) may measure the pressure in the chamber 101 as an explosion factor. The pressure measurement unit 182 may output the measurement result to the operation controller 185. To this end, the pressure measurement unit 182 may be in communication with the chamber 101. If the pressure in the chamber 101 is lower than the external pressure, an explosive gas may flow in from outside the chamber 101 into the chamber 101. This may cause the apparatus 100 to explode. Thus, the pressure in the chamber 101 may correspond to the explosion factor. For example, supplying an inert gas or fresh air may be possible to prevent the presence of an explosive atmosphere in the chamber 101 and/or to prevent infiltration of an external explosive atmosphere via/by internal pressurization.

The discharge port 183 may serve as an outlet configured to discharge a fluid from the chamber 101 to outside of the chamber 101 via an output terminal OUT2. The discharge port 183 may be one of the components of the explosion-prevention unit 180A that is disposed in the chamber 101. As such, at least part (e.g., the discharge port 183) of the explosion-prevention unit 180 may be disposed in the chamber 101.

The detection unit 184 (e.g., hydrogen detector) may detect whether hydrogen is included in the fluid discharged to the outside of the chamber 101 via the discharge port 183. The detection unit 184 may output the detection result to the operation controller 185.

Based on at least one of the pressure measured by the pressure measurement unit 182 or the result of the detection by the detection unit 184, the operation controller 185 may stop the operation of at least one of the gas analysis unit 140, the target selection unit 110, the standard gas supply unit 120, the gas selection unit 130, or the discharge pump 150 (e.g., which may be disposed in the chamber 101). For example, if the detection unit 184 detects that hydrogen is included in the fluid discharged from the discharge port 183, the operation controller 185 may output a warning message via the notification unit 186 (e.g., interface) to prevent explosion of the apparatus 100. The operation controller 185 may perform a shut-down operation that automatically and/or manually shuts off various electrically driven actuators and/or valves in the chamber 101.

The notification unit 186 may output an explosion warning message in various ways, such as visually and/or audibly (e.g., via a screen and/or a speaker), under the control of the operation controller 185.

The fluid supply unit 187 may supply dry air or nitrogen (N₂) into the chamber 101 to form a positive pressure inside the chamber 101 to satisfy the pressure explosion-prevention condition and/or may maintain the positive pressure to prevent the presence of an explosive atmosphere in the chamber 101. In this way, the explosion-prevention conditions may be satisfied by pressure control. The explosion-prevention conditions may be satisfied by other methods, such as using a pressure-resistant structure and/or an intrinsically safe design. However, the examples are not limited to any specific method of satisfying the explosion-prevention conditions.

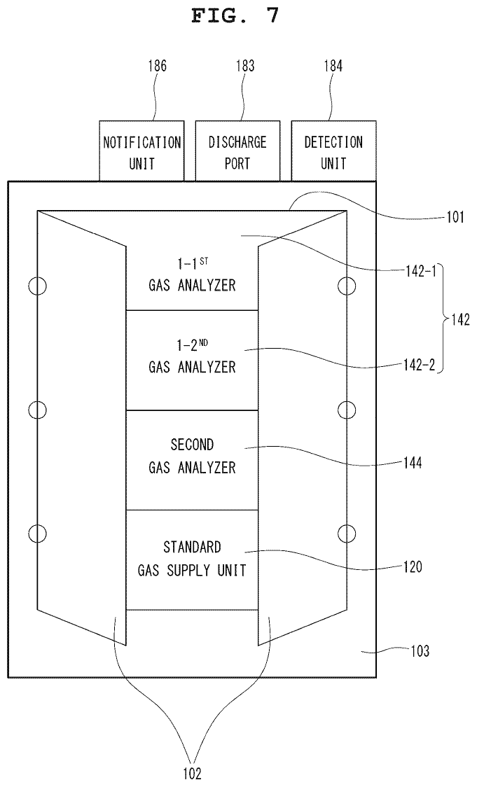

FIG. 7 is a front view showing the external appearance of the hydrogen quality analysis and management apparatus 100 according to the example.

According to an example, inside the chamber 101, the second gas analyzer 144 may be disposed above the standard gas supply unit 120, the 1-1st and 1-2^{nd} gas analyzers 142-1 and 142-2 may be disposed above the second gas analyzer 144. The detection unit 184, the discharge port 183, and the notification unit 186, which may be components of the explosion-prevention unit 180, may be disposed above the first and second gas analyzers 142 and 144.

Referring back to FIG. 1, the controller 200 may control the overall operation of the hydrogen quality analysis and management apparatus 100 according to the example.

The controller 200 may include a communication device communicating with other controllers and/or sensor(s) to control one or more functions and/or operations in charge, a memory storing an operation system, a logic command, and input/output information, and/or one or more processors for performing one or more determinations, calculations, and/or decisions necessary for controlling the function in charge. A controller may include, for example, a processor (e.g., a central processing unit (CPU)), a microchip, a logic, an application-specific integrated circuit (ASIC), memory, etc. A controller may manipulate and/or control other components in the system (e.g., vehicle).

The controller 200 may include a quality determination unit 210. The controller 200 may further include a quality management unit 220 and a process controller 230. The controller 200 may further include a calibration unit 240. The quality determination unit 210, the quality management unit 220, and the calibration unit 240 may generally refer to items that logically can be grouped together to perform a function or group of related functions as described herein. The quality determination unit 210, the quality management unit 220, and the calibration unit 240 may be implemented in software, hardware and/or a combination of software and hardware. The quality determination unit 210, the quality management unit 220, and the calibration unit 240 functions described above may be implemented and/or performed by one or more processors (e.g., of the controller 200 and/or controlled by the controller 200). For examples, the quality determination unit 210, the quality management unit 220, and the calibration unit 240 may include processor(s), microprocessor(s), graphics processing unit(s), logic circuit(s), dedicated circuit(s), application-specific integrated circuit(s), programmable array logic, field-programmable gate array(s), controller(s), microcontroller(s), and/or other suitable hardware. The quality determination unit 210, the quality management unit 220, and the calibration unit 240 may also include software control module(s) implemented with a processor or logic circuitry for example. The quality determination unit 210, the quality management unit 220, and the calibration unit 240 may include or otherwise be able to access memory such as, for example, one or more non-transitory computer-readable storage media, such as random-access memory, read-only memory, electrically erasable programmable read-only memory, erasable programmable read-only memory, flash/other memory device(s), data registrar(s), database(s), and/or other suitable hardware. One or more storage type media may include any or all of the tangible memory of computers, processors, or the like, or associated modules thereof, such as various semiconductor memories, tape drives, disk drives and the like, which may provide non-transitory storage at any time for software programming.

The quality determination unit 210 may determine the suitability of the quality of the target gas using the results of the analysis by the gas analysis unit 140 (step 330). For example, the quality determination unit 210 may be configured to receive, from the first and second gas analyzers 142-1, 142-2, and 144, detected levels of various impurities.

For example, if the gas analysis unit 140 measures the concentration of ten impurity components as described above, the quality determination unit 210 may determine that the quality of the target gas is unsuitable if the concentration of any impurity component exceeds the reference concentration specified in Table 1 below.

TABLE 1

| | | Reference | |
|---|---|---|---|
| | | Concentration | Detection Limit |
| Classification | Component | (ppm) | (ppm) |
| 1 | CH₄ | 2 | 0.05 |
| 2 | CO | 0.2 | 0.005 |
| 3 | HCHO | 0.01 | 0.001 |
| 4 | NH₃ | 0.1 | 0.005 |
| 5 | H₂O | 5 | 0.1 |
| 6 | CO₂ | 2 | 0.02 |
| 7 | H₂S | 0.004 | 0.001 |
| 8 | HCOOH | 0.2 | 0.01 |
| 9 | O₂ | 5 | 0.5 |
| 10 | N₂ | 300 | 0.1 |

The detection limit in Table 1 represents the minimum unit of the concentration measurable by the gas analysis unit 140, that is, the resolution (or resolving power).

The calibration unit 240 may calibrate the first and second gas analyzers 140 (142-1, 142-2, and 144) using the results of the analysis by the first and second gas analyzers 140 (142-1, 142-2, and 144) after the plurality of standard gases is supplied to the first and second gas analyzers 140 (142-1, 142-2, and 144). For example, after supplying the plurality of standard gases to the first and second gas analyzers 140 (142-1, 142-2, and 144), if it is necessary to adjust the concentration of the standard gases, the calibration unit 240 may perform calibration by controlling the standard gas supply unit 120 using a selection signal SS to adjust the concentration of the standard gases.

Also, or alternatively, the hydrogen quality analysis and management apparatus may not include the calibration unit 240, the standard gas supply unit 120, and the gas selection unit 130. For example, the target gas selected by the target selection unit 110 may be directly supplied to the gas analysis unit 140.

The quality management unit 220 may receive, manage, and store data related to the suitability of the quality determined by the quality determination unit 210. Accordingly, the quality of the target gases at the points T1, T2, T3, and T4 during the process may be stored and managed over an extended period.

The process controller 230 may monitor the data managed by the quality management unit 220 and/or may control the operation of at least a portion of the process from hydrogen production to shipment.

For example, the process controller 230 may implement normal operation (e.g., on-site), which may require/specify inspection of the suitability of the quality of the first, second, and third target gases, so that the produced hydrogen may be shipped to a remote location and/or may be supplied to the dispenser 570.

The process controller 230 may implement shipment operation, which may require/specify inspection of the suitability of the quality of the first and second target gases, so that the produced hydrogen may be shipped to a remote location.

The process controller 230 may implement abnormal operation (e.g., off-site), which may require/specify inspection of the suitability of the quality of the third and fourth target gases, so that hydrogen supplied from the backup hydrogen supply unit 580 may be supplied to the dispenser 570.

For the above-described operation, if the quality of the second target gas is determined to be suitable, the process controller 230 may generate a control signal via an output terminal OUT4, so that hydrogen stored in the low-pressure storage unit 540 may be discharged to the high-pressure compressor 550 and/or may be shipped via a tube trailer to be supplied to a remote location.

If the quality of the third target gas is determined to be suitable, the process controller 230 may generate a control signal and send via the output terminal OUT4, so that hydrogen stored in the high-pressure storage unit 560 may be supplied to a hydrogen vehicle through the dispenser 570.

For example, if the quality of the fourth target gas is determined to be suitable, the process controller 230 may generate a control signal and send via the output terminal OUT4, so that hydrogen stored in the backup hydrogen supply unit 580 may be supplied to the high-pressure compressor 550 through the second buffer tank 590.

The apparatus 100 and the method 300 for analyzing and managing hydrogen quality may selectively operate with three or fewer hydrogen quality measurement points, rather than with four points as discussed in the example herein. Operating with fewer measurement points may be performed depending on the operating conditions of the reformer and the hydrogen refueling station, and may enabling efficient hydrogen quality management while reducing time and energy consumption.

For example, the process controller 230 may be implemented as a server or a computer (e.g., personal computer (PC)).

The apparatus 100 and the method 300 for analyzing and managing hydrogen quality according to the examples may measure and manage hydrogen quality (e.g., in real time and/or near real time) throughout the process discussed herein (e.g., including production, compression, storage, and charging of hydrogen), thereby ensuring that hydrogen is supplied with high reliability and stability. As a result, it may be possible to contribute to the stable supply of hydrogen and the expansion of hydrogen vehicle adoption by enabling immediate responses to issues related to hydrogen quality, for example, by monitoring hydrogen quality in real time to prevent the supply of defective hydrogen.

In general, in a hydrogen refueling station according to a comparative example, when measuring the concentration of impurity components, other than hydrogen, included in the target gas, six impurity components (CH₄, CO, CO₂, N₂, O₂, and H₂O) are measured using analysis methods, such as gas chromatography (GC) or cavity ring-down spectroscopy (CRDS). In such a comparative example, the number of impurity components that are measurable is limited, and it is also difficult to perform real-time continuous measurement. This is because, for example, when using the GC analysis method, it takes at least 10 minutes to measure N₂ and O₂ once. As a result, real-time continuous measurement is not feasible under actual field operating conditions, and some impurity components with a high probability of detection may not be properly managed, potentially causing problems in hydrogen quality management.

In contrast, since the apparatus 100, according to the example, uses OFCEAS, which is capable of continuously measuring the concentration of impurity components over time, it may be possible to analyze the concentration of impurity components contained in the target gas including hydrogen in real time.

The hydrogen quality analysis and management apparatus and method according to the above-described examples may optimize the analysis operation according to the operating conditions of the reformer and the hydrogen refueling station, thereby achieving high efficiency. The apparatus and the method according to the examples may manage the quality of ten impurity components based on ISO 14697, thereby analyzing hydrogen quality in real time with high reliability.

According to the example, when the target selection unit 110 supplies a target gas to the gas analysis unit 140 in order to measure the concentration of impurity components contained in each of a plurality of target gases, one target gas may first be supplied to the gas analysis unit 140, followed by a purge gas and then another target gas, whereby contamination between the target gases to be subjected to purity measurement may be prevented.

According to the example, the accuracy and reliability of the gas analysis unit 140 may be secured by periodically inspecting and calibrating the gas analysis unit 140 using standard sample gases STD1 and STD2.

By selecting and measuring gases to be measured based on the operating conditions of the reformer and the hydrogen refueling station, hydrogen quality management may be performed efficiently. By measuring a plurality of impurity components in real time and by monitoring and managing hydrogen quality, the supply of hydrogen with quality issues to the market may be prevented. Thus, proactive management of hydrogen quality may be achieved.

A hydrogen quality analysis and management apparatus according to an example may include a first gas analyzer configured to analyze the concentration of a plurality of impurity components contained in a target gas including hydrogen to be analyzed using first and second laser analysis methods, a second gas analyzer configured to analyze the concentration of the plurality of impurity components using a non-laser analysis method, and a quality determination unit configured to determine the suitability of quality of the target gas based on the results of the analysis by the first and second gas analyzers.

In an example, the hydrogen quality analysis and management apparatus may further include a target selection unit configured to output the target gas selected at a predetermined time point during a process, including hydrogen production, storage after compression, backup supply, and charging, to the first and second gas analyzers.

In an example, the target gas may include a plurality of target gases, and the plurality of target gases may include at least two of a first target gas including produced hydrogen, a second target gas including hydrogen compressed to a low pressure after being produced, a third target gas including hydrogen compressed to a high pressure after being compressed to the low pressure, or a fourth target gas including backup hydrogen.

In an example, the target selection unit may select at least one of first to N^{th} (N being a positive integer of 2 or greater) target gases or may sequentially or alternately discharge the selected target gases.

In an example, the target selection unit may include first to N^{th} filters, first to N^{th} regulators, first to N^{th} control valves, and first to N^{th} check valves. An n^{th} (1≤n≤N) filter may filter contaminants contained in an n^{th} target gas, an n^{th} regulator may regulate the pressure of the n^{th} target gas filtered by the n^{th} filter, an n^{th} control valve may allow or restrict the flow of the n^{th} target gas output from the n^{th} regulator based on an n^{th} opening/closing control signal, and an n^{th} check valve may prevent backflow of the n^{th} target gas toward the n^{th} control valve.

In an example, after at least one of the first to N^{th} target gases is discharged to the first and second gas analyzers, the target selection unit may discharge a purge gas to the first and second gas analyzers before at least another of the first to N^{th} target gases is discharged to the first and second gas analyzers.

In an example, the target selection unit may further include an N+1^{th} filter configured to filter contaminants contained in the purge gas, an N+1^{th} regulator configured to regulate the pressure of the purge gas filtered by the N+1^{th} filter, an N+1^{th} control valve configured to allow or restrict the flow of the purge gas output from the N+1^{th} regulator based on an N+1^{th} opening/closing control signal, and an N+1^{th} check valve configured to prevent backflow of the purge gas toward the N+1^{th} control valve.

In an example, the hydrogen quality analysis and management apparatus may further include at least two of a first pipe connected between a reformer, configured to produce hydrogen, and the target selection unit to pass the first target gas, a second pipe connected between a low-pressure storage unit, configured to store the hydrogen produced by the reformer and compressed to a low pressure, and the target selection unit to pass the second target gas, a third pipe connected between a high-pressure storage unit, configured to store the second target gas compressed to a high pressure, and the target selection unit to pass the third target gas, or a fourth pipe connected between a backup hydrogen supply unit and the target selection unit to pass the fourth target gas.

In an example, the first gas analyzer may analyze the concentration of six impurity components, including CO₂, NH₃, HCOOH, HCHO, CO, and CH₄, among the plurality of impurity components, using optical feedback cavity enhanced absorption spectroscopy (OFCEAS) as the first laser analysis method, and may analyze the concentration of two impurity components, including H₂S and H₂O, among the plurality of impurity components, using OFCEAS as the second laser analysis method, and the second gas analyzer may analyze the concentration of two impurity components, including N₂ and O₂, among the plurality of impurity components, using gas chromatography (GC) as the non-laser analysis method.

In an example, the hydrogen quality analysis and management apparatus may further include a standard gas supply unit configured to supply a plurality of standard gases, each having standard concentration of the plurality of impurity components, and a gas selection unit configured to supply an output from one of the target selection unit and the standard gas supply unit to the first and second gas analyzers based on a selection control signal.

In an example, the standard gas supply unit may include a mass flow controller configured to adjust the concentration of the plurality of standard gases and to supply the plurality of standard gases having the adjusted concentration to the gas selection unit.

In an example, the hydrogen quality analysis and management apparatus may further include a calibration unit configured to calibrate the first and second gas analyzers based on the results of the analysis by the first and second gas analyzers after the plurality of standard gases is supplied to the first and second gas analyzers.

In an example, the hydrogen quality analysis and management apparatus may further include a discharge pump configured to pump and discharge the target gas analyzed by the first and second gas analyzers and to form a vacuum inside the first gas analyzer.

In an example, the hydrogen quality analysis and management apparatus may further include a chamber configured to accommodate at least one of the first gas analyzer, the second gas analyzer, the target selection unit, the standard gas supply unit, the gas selection unit, or the discharge pump and to satisfy explosion-prevention conditions.

In an example, the hydrogen quality analysis and management apparatus may further include an explosion-prevention unit configured to inspect an explosion factor in the chamber and to perform an operation associated with the explosion factor based on the inspection result.

In an example, the explosion-prevention unit may include a pressure measurement unit configured to measure the pressure in the chamber as the explosion factor, a discharge port configured to discharge a fluid in the chamber, a detection unit configured to detect whether hydrogen is included in the fluid discharged through the discharge port, an operation controller configured to stop operation of the first and second gas analyzers, the target selection unit, the standard gas supply unit, the gas selection unit, or the discharge pump, disposed in the chamber, based on at least one of the measured pressure or the result of the detection by the detection unit, and a notification unit configured to output an explosion warning message under the control of the operation controller.

In an example, the explosion-prevention unit may further include a fluid supply unit configured to supply dry air or nitrogen into the chamber to form a positive pressure inside the chamber to satisfy the explosion-prevention conditions and configured to maintain the positive pressure to prevent the presence of an explosive atmosphere in the chamber.

In an example, the first and second gas analyzers may be disposed above the standard gas supply unit, and the explosion-prevention unit may be disposed above the first and second gas analyzers.

In an example, the hydrogen quality analysis and management apparatus may further include a quality management unit configured to receive, manage, and store data associated with the suitability of the quality determined by the quality determination unit and a process controller configured to monitor the data to control an operation of at least a portion of a process from hydrogen production to shipment.

A hydrogen quality analysis and management apparatus according to another example may include a target selection unit configured to select a target gas including hydrogen to be analyzed at a predetermined time point during a process, including hydrogen production, storage after compression, backup supply, and charging, a gas analysis unit configured to analyze the concentration of a plurality of impurity components contained in the target gas selected by the target selection unit, and a quality determination unit configured to determine the suitability of quality of the target gas based on the result of the analysis by the gas analysis unit.

In an example, the gas analysis unit may include a 1-1st gas analyzer configured to analyze the concentration of six impurity components, including CO₂, NH₃, HCOOH, HCHO, CO, and CH₄, among the plurality of impurity components, using optical feedback cavity enhanced absorption spectroscopy (OFCEAS) as a first laser analysis method, a 1-2^{nd} gas analyzer configured to analyze the concentration of two impurity components, including H₂S and H₂O, among the plurality of impurity components, using OFCEAS as a second laser analysis method, and a second gas analyzer configured to analyze the concentration of two impurity components, including N₂ and O₂, among the plurality of impurity components, using gas chromatography (GC) as a non-laser analysis method.

A hydrogen quality analysis and management method according to still another example may include selecting a target gas including hydrogen to be analyzed during a process, including hydrogen production, storage after compression, backup supply, and charging, analyzing the concentration of a plurality of impurity components contained in the selected target gas, and determining the suitability of quality of the target gas based on the result obtained in the analyzing.

The hydrogen quality analysis and management apparatus and method according to the examples may be applied to hydrogen production infrastructure, hydrogen refueling infrastructure, or fuel cell apparatuses.

As is apparent from the above description, the hydrogen quality analysis and management apparatus and method according to the examples may measure and manage hydrogen quality in real time throughout the entire process, may analyze the concentration of impurity components contained in the target gas including hydrogen in real time, and may prevent contamination between the target gases to be subjected to purity measurement. Furthermore, since the accuracy and reliability of the gas analysis unit are secured, it may be possible to contribute to the reliable and stable supply of hydrogen and the expansion of hydrogen vehicle adoption by monitoring hydrogen quality in real time to prevent the supply of defective hydrogen.

However, the effects achievable through the disclosure are not limited to the above-mentioned effects, and other effects not mentioned herein will be clearly understood by those skilled in the art from the above description.

The above-described various examples may be combined with each other without departing from the scope of the present disclosure unless they are incompatible with each other.

For any element or process that is not described in detail in any of the various examples, reference may be made to the description of an element or a process having the same reference numeral in another example, unless otherwise specified.

While the present disclosure has been particularly shown and described with reference to examples thereof, these examples are only proposed for illustrative purposes, and do not restrict the present disclosure, and it will be apparent to those skilled in the art that various changes in form and detail may be made without departing from the essential characteristics of the examples set forth herein. For example, respective configurations set forth in the examples may be modified and applied. Further, differences in such modifications and applications should be construed as falling within the scope of the present disclosure as defined by the appended claims.

What is claimed is:

1. An apparatus comprising:
a first gas analyzer comprising at least one laser absorption spectrometer configured to:
measure one or more first concentrations of one or more first impurity components in a target gas comprising hydrogen; and
measure one or more second concentrations of one or more second impurity components in the target gas;
a second gas analyzer configured to measure, via a non-laser-based sensing method, one or more third concentrations of one or more third impurity components in the target gas; and
a controller comprising:
a processor; and
a memory storing instructions that, when executed by the processor, are configured to cause the apparatus to:
receive, from the first gas analyzer, first information associated with the one or more first concentrations and second information associated with the one or more second concentrations; and
receive, from the second gas analyzer, third information associated with the one or more third concentrations;
determine, based on the first information, the second information and the third information, an indicator of a quality of the target gas; and
control, based on the determined indicator of the quality, a process associated with supply of hydrogen.

2. The apparatus according to claim 1, further comprising a first gas selection valve system configured to cause selective output, to the first gas analyzer and the second gas analyzer, of the target gas collected at one or more stages of the process, wherein the process comprises stages of: hydrogen production, compressed hydrogen storage, hydrogen backup supply, and hydrogen charging.

3. The apparatus according to claim 2, wherein the target gas comprises one of a plurality of target gases collected at a plurality of the stages of the process, and
wherein the plurality of target gases comprises at least two of:
a first target gas collected at the hydrogen production stage;
a second target gas comprising hydrogen compressed to a first pressure after being produced;
a third target gas comprising hydrogen compressed to a second pressure higher than the first pressure; or
a fourth target gas comprising backup hydrogen stored as backup for the hydrogen compressed to the second pressure.

4. The apparatus according to claim 3, wherein the plurality of target gases comprises N target gases, wherein the first gas selection valve system is configured to cause the selective output of: one of a first to an N^{th} target gas, or two or more of the plurality of target gases or sequentially or alternately.

5. The apparatus according to claim 4, wherein the first gas selection valve system comprises:
first to N^{th} filters;
first to N^{th} regulators;
first to N^{th} control valves; and
first to N^{th} check valves,
wherein an i^{th} (1≤i≤N) filter is configured to filter contaminants from an i^{th} target gas of the plurality of target gases,
wherein an i^{th} regulator is configured to regulates a pressure of the i^{th} target gas filtered by the i^{th} filter,
wherein an i^{th} control valve is configured to control, based on an i^{th} opening/closing control signal, a flow of the i^{th} target gas output from the i^{th} regulator, and
wherein an i^{th} check valve is configured to prevent backflow of the i^{th} target gas toward the i^{th} control valve.

6. The apparatus according to claim 4, wherein the first gas selection valve system is further configured to cause output, to the first gas analyzer and the second gas analyzer, of a purge gas between selective outputs at least two of the first to N^{th} target gases.

7. The apparatus according to claim 6, wherein the first gas selection valve system further comprises:
an N+1^{th} filter configured to filter contaminants from the purge gas;
an N+1^{th} regulator configured to regulate a pressure of the purge gas filtered by the N+1^{th} filter;
an N+1^{th} control valve configured to control, based on an N+1^{th} opening/closing control signal, a flow of the purge gas output from the N+1^{th} regulator; and
an N+1^{th} check valve configured to prevent backflow of the purge gas toward the N+1^{th} control valve.

8. The apparatus according to claim 3, further comprising at least two of:
a first pipe connected between a reformer and the first gas selection valve system, wherein the reformer is configured to produce the first target gas comprising hydrogen;
a second pipe connected between a low-pressure storage and the first gas selection valve system, wherein the low-pressure storage is configured to compress the first target gas produced by the reformer to the first pressure to form the second target gas;
a third pipe connected between a high-pressure storage and the first gas selection valve system, wherein the high-pressure storage is configured to compress the second target gas to the second pressure to form the third target gas; or
a fourth pipe connected between a backup hydrogen supply and the first gas selection valve system, wherein the backup hydrogen supply is configured to store the fourth target gas.

9. The apparatus according to claim 1, wherein a first laser absorption spectrometer of the at least one laser absorption spectrometer is configured to use optical feedback cavity enhanced absorption spectroscopy (OFCEAS) to measure concentrations of CO₂, NH₃, HCOOH, HCHO, CO, and CH₄ of the one or more first impurity components,
wherein a second laser absorption spectrometer of the at least on laser absorption spectrometer is configured to use OFCEAS to measure concentrations of H₂S and H₂O of the one or more second impurity components, and
wherein the second gas analyzer is configured to use gas chromatography (GC) to measure the concentrations of N₂ and O₂ of the one or more third impurity components.

10. The apparatus according to claim 2, further comprising:
a standard gas supply configured to supply a plurality of standard gases, each having standard concentration of at least one impurity component of a plurality of impurity components comprising: the one or more first impurity components, the one or more second impurity components, and the one or more third impurity components; and
a second gas selection valve system configured to, based on a selection control signal, supply, to the first gas analyzer and the second gas analyzer, an output from one of the first gas selection valve system and the standard gas supply.

11. The apparatus according to claim 10, wherein the standard gas supply comprises a mass flow controller configured to adjust concentrations of the plurality of standard gases and to supply the plurality of standard gases having the adjusted concentrations to the second gas selection valve system.

12. The apparatus according to claim 10, wherein the controller is configured to:
receive, from the first gas analyzer and the second gas analyzer, information indicating one or more concentrations of one or more of the plurality of impurity components in the plurality of standard gases; and
calibrate, based on the information indicating the one or more concentrations of the one or more of the plurality of impurity components, the first gas analyzer and the second gas analyzer.

13. The apparatus according to claim 10, further comprising a discharge pump configured to pump and discharge the target gas from the first gas analyzer to form a vacuum inside the first gas analyzer.

14. The apparatus according to claim 13, further comprising a chamber configured to accommodate at least one of the first gas analyzer, the second gas analyzer, the first gas selection valve system, the standard gas supply, the second gas selection valve system, or the discharge pump and to satisfy explosion-prevention conditions.

15. The apparatus according to claim 14, further comprising an explosion-prevention unit comprising:
a pressure sensor configured to measure a pressure in the chamber;
a discharge port configured to discharge a fluid from the chamber;
a hydrogen detector configured to detect whether hydrogen is in the fluid discharged via the discharge port;
an operation controller configured to, based on at least one of the measured pressure or a detection by the hydrogen detector, stop operation of the first gas analyzer and the second gas analyzer, the first gas selection valve system, the standard gas supply, the second gas selection valve system, or the discharge pump; and
an interface configured to, based on control by the controller, output an explosion warning message.

16. The apparatus according to claim 15, wherein the explosion-prevention unit further comprises a fluid supply configured to:
supply air or nitrogen into the chamber to form a positive pressure inside the chamber to satisfy the explosion-prevention conditions, and
maintain the positive pressure to prevent an explosive atmosphere in the chamber.

17. An apparatus comprising:
a first gas selection valve system configured to select a target gas comprising hydrogen to be analyzed, wherein the target gas is collected at one or more stages of a process associated with supply of hydrogen, wherein the process comprises stages of hydrogen production, compressed hydrogen storage, hydrogen backup supply, and hydrogen charging;
a plurality of gas analyzers configured to measure concentrations of a plurality of impurity components in the target gas selected by the first gas selection valve system; and
a control circuit configured to cause the apparatus to:
receive, from the plurality of gas analyzers, information indicating one or more concentrations of one or more impurity components of the plurality of impurity components;
determine, based on the information, an indicator of a quality of the target gas; and
control, based on the determined indicator of the quality, the process associated with the supply of hydrogen.

18. The apparatus according to claim 17, wherein the plurality of gas analyzers comprises:
a first gas analyzer comprising at least one laser absorption spectrometer configured to:
measure one or more first concentrations of one or more first impurity components in the target gas; and
measure one or more second concentrations of one or more second impurity components in the target gas; and
a second gas analyzer configured to measure, via a non-laser-based sensing method, one or more third concentrations of one or more third impurity components.

19. A method performed by an apparatus for analyzing and managing hydrogen quality, the method comprising:
controlling flow of a target gas, of a plurality of target gases, to a plurality of gas analyzers configured to measure concentrations of a plurality of impurity components, wherein the plurality of target gases comprises hydrogen to be analyzed and each target gas of the plurality of target gases is collected at a different stage of a process associated with hydrogen supply, wherein the process comprises stages of hydrogen production, compressed hydrogen storage, hydrogen backup supply, and hydrogen charging;
receiving, from the plurality of gas analyzers, information indicating one or more concentrations of one or more impurity components of the plurality of impurity components;
determining, based on the information, an indicator of a quality of the target gas; and
controlling, based on the determined indicator of the quality, the process associated with the supply of hydrogen.

20. The method according to claim 19, wherein the plurality of gas analyzers comprises:
a first gas analyzer comprising at least one laser absorption spectrometer configured to:
measure one or more first concentrations of one or more first impurity components in the target gas; and
measure one or more second concentrations of one or more second impurity components in the target gas; and
a second gas analyzer configured to measure, via a non-laser-based sensing method, one or more third concentrations of one or more third impurity components.
